(19) United States
(12) Patent Application Publication
(10) Pub. No.: US 2012/0070339 A1
(43) Pub. Date: Mar. 22, 2012

(54) ULTRAVIOLET DISINFECTION OF OIL FIELD PROCESS WATER

(75) Inventor: Oliver Lawal, Walton, KY (US)

(73) Assignee: AQUIONICS, INC., Erlanger, KY (US)

(21) Appl. No.: 13/276,498

(22) Filed: Oct. 19, 2011

Related U.S. Application Data
(63) Continuation-in-part of application No. PCT/US2011/029984, filed on Mar. 25, 2011.
(60) Provisional application No. 61/318,086, filed on Mar. 26, 2010.

(51) Int. Cl. A61L 2/10; B01J 19/12

(52) U.S. Cl. 422/24; 422/186.3

(57) ABSTRACT

Methods and systems for inactivating Desulfovibrio desulfuricans in a fracturing fluid are disclosed. The methods include exposing the fracturing fluid to a dose of from about 4 mJ/cm² to about 10 mJ/cm² of polychromatic ultraviolet radiation. The polychromatic ultraviolet radiation includes a plurality of major inactivation wavelength peaks in a range of from about 200 nm to about 400 nm. The system includes an ultraviolet radiation chamber in fluid communication with a fracturing fluid source and a wellbore, and at least one medium pressure ultraviolet lamp arranged substantially within the ultraviolet radiation chamber. The medium pressure ultraviolet lamp exposes the fracturing fluid containing the Desulfovibrio desulfuricans to a dose of from about 4 mJ/cm² to about 10 mJ/cm² of polychromatic ultraviolet radiation.

The present application is filed under 35 U.S.C. §111(a) as a continuation-in-part of International Patent Application No. PCT/US2011/029984 (AQU 0002 PB), which international application designates the United States and claims the benefit of U.S. Provisional Application Ser. No. 61/318,086 (AQU 0002 MA), filed Mar. 26, 2010.

### TECHNICAL FIELD

The present disclosure relates to methods and systems for inactivating anaerobic sulfate-reducing bacteria in a fluid using ultraviolet radiation. More specifically, the present disclosure relates to methods and systems for inactivating Desulfovibrio desulfuricans in a fracturing fluid using a dose of polychromatic ultraviolet radiation.

### BACKGROUND

Fracturing fluids in oilfield installations generally require treatment to reduce aerobic acid-producing bacteria and anaerobic sulfate-reducing bacteria. The removal of sulfate-reducing bacteria can prevent a variety of stimulation problems. For example, the removal of sulfate-reducing bacteria combats microbe induced corrosion and prevents iron sulfide precipitation and souring of the reservoir with hydrogen sulfide gas.

Currently, oxidizing and non-oxidizing biocides are used to reduce aerobic acid-producing bacteria and anaerobic sulfate-reducing bacteria. However, the use of oxidizing biocides is imperfect in that biocides may oxidize not only the cells of bacteria but also polymers present in the fracturing fluids, leading to increased pressures and decreased viscosity. The use of non-oxidizing biocides is also imperfect in that the biocides may hydrate polymers present in the fracturing fluids, leading to loss of fluid stability. Biocides are also imperfect in that determining the quantity required for maximum efficacy is difficult. Moreover, the use of biocides leads to a variety of regulatory concerns, including health and safety in transport and handling, and environmental concerns, including limiting the use of fresh water and chemical treatment. Accordingly, additional embodiments for methods and systems for inactivating anaerobic sulfate-reducing bacteria in fracturing fluids are desired.

### SUMMARY

The present disclosure is based on the discovery that polychromatic ultraviolet (hereinafter “UV”) radiation inactivates the anaerobic sulfate-reducing bacteria Desulfovibrio desulfuricans (hereinafter “D. desulfuricans”). D. desulfuricans is a species of anaerobic sulfate-reducing bacteria in the Desulfovibrionaceae family, commonly found in water. Accordingly, in one embodiment, a method for inactivating D. desulfuricans in a fracturing fluid containing the D. desulfuricans is disclosed. The method includes exposing the fracturing fluid containing the Desulfovibrio desulfuricans to a dose of from about 4 mJ/cm² to about 10 mJ/cm² of polychromatic UV radiation. The polychromatic UV radiation includes a plurality of major inactivation wavelength peaks in a range of from about 200 nm to about 400 nm. The major inactivation wavelength peaks are characterized by an intensity greater than about 25% a maximum peak intensity of the plurality of major inactivation wavelength peaks and by a full width half maximum value greater than about 2 nanometers. The dose of polychromatic ultraviolet radiation inactivates the Desulfovibrio desulfuricans.

In another embodiment, a method for inactivating D. desulfuricans in a fracturing fluid containing the D. desulfuricans is disclosed. The method includes exposing the fracturing fluid containing the D. desulfuricans to a dose of from about 4 mJ/cm² to about 10 mJ/cm² of polychromatic UV radiation. The fracturing fluid is water and flows substantially unidirectionally from a fracturing fluid source to wellbore. The polychromatic UV radiation includes at least five inactivation wavelength peaks in the range of from about 200 nm to about 400 nm. The inactivation wavelength peaks are characterized an intensity greater than about 25% of a maximum peak intensity of the plurality of major inactivation wavelength peaks and by a full width half maximum value greater than about 2 nanometers. The dose of polychromatic UV radiation inactivates the D. desulfuricans.

In yet another embodiment, a system for inactivating D. desulfuricans in a fracturing fluid containing the D. desulfuricans is disclosed. The system includes an UV radiation chamber and at least one medium pressure UV lamp. The medium pressure UV lamp is arranged substantially within the UV radiation chamber. The UV radiation chamber is in fluid communication with a fracturing fluid source and a wellbore. The fracturing fluid flows substantially unidirectionally from the fracturing fluid source through the ultraviolet radiation chamber to the wellbore. The medium pressure UV lamp exposes the fracturing fluid containing the D. desulfuricans to a dose of from about 4 mJ/cm² to about 10 mJ/cm² of polychromatic UV radiation. The polychromatic UV radiation includes a plurality of major inactivation wavelength peaks in the range of from about 200 nm to about 400 nm. The major inactivation wavelength peaks are characterized by an intensity greater than about 25% a maximum peak intensity of the plurality of major inactivation wavelength peaks and by a full width half maximum value greater than about 2 nanometers. The dose of polychromatic UV radiation inactivates the D. desulfuricans.

These and other features and advantages of these and other various embodiments according to the present disclosure will become more apparent in view of the drawings, detailed description, and claims provided herein.

### BRIEF DESCRIPTION OF THE SEVERAL DRAWINGS

The following detailed description of the embodiments of the present disclosure can be better understood when read in conjunction with the following drawings, where like structure is indicated with like reference numerals, and in which:

FIG. 1 is a graph of a typical UV output spectrum of wavelength (nm) of a medium pressure UV lamp;

FIG. 2 is a perspective view of a system for inactivating D. desulfuricans in a fracturing fluid according to an embodiment of the present disclosure; and

FIG. 3 is a graph of the response of D. desulfuricans to UV dose (mJ/cm²) irradiated with medium pressure UV with respect to log inactivation.

Skilled artisans appreciate that elements in the figures are illustrated for simplicity and clarity and are not necessarily drawn to scale. For example, the dimensions of some of the elements in the figures may be exaggerated relative to other elements, as well as conventional parts removed, to help to improve understanding of the various embodiments of the present disclosure.

### DETAILED DESCRIPTION

The following terms are used in the present application:

As used herein, the term “fracturing fluid” refers to a fluid which may be employed in hydraulic fracturing to initiate a fracture in a reservoir rock formation, to propagate a fracture in a reservoir rock formation, and/or to transport a proppant along the length of a fracture in a reservoir rock formation. For example, in the context of hydraulic fracturing, a wellbore is drilled into a reservoir rock formation, and a fracturing fluid is pumped into the wellbore in order to release and/or facilitate release of petroleum, natural gas, coal seam gas, and/or other substances for extraction from the reservoir rock formation. In one particular example, the fracturing fluid is water.

As used herein, the term “dose” refers to a quantity of energy of polychromatic UV light.

As used herein, the term “wellbore” refers to a hole drilled in a reservoir rock formation for the purpose of extracting petroleum, natural gas, coal seam gas, and/or other substances therefrom.

I. Method for Inactivating D. desulfuricans

Embodiments of the present disclosure relate to methods and systems for inactivating D. desulfuricans in a fracturing fluid containing the D. desulfuricans. In one embodiment, a method for inactivating D. desulfuricans in a fracturing fluid containing the D. desulfuricans is disclosed. The method includes exposing the fracturing fluid containing the D. desulfuricans to a dose of from about 4 mJ/cm² to about 10 mJ/cm² of polychromatic UV radiation. The polychromatic UV radiation includes a plurality of major inactivation wavelength peaks in a wavelength range of from about 200 nm to about 400 nm. Each of the major inactivation wavelength peaks is characterized by an intensity greater than about 25% a maximum peak intensity of the plurality of major inactivation wavelength peaks and by a full width half maximum value greater than about 2 nanometers. The dose of polychromatic ultraviolet radiation inactivates the D. desulfuricans.

The fracturing fluid containing D. desulfuricans is exposed to a dose of polychromatic UV radiation of from about 4 mJ/cm² to about 10 mJ/cm², or alternatively of from about 6 mJ/cm² to about 10 mJ/cm², or alternatively of from about 7.5 mJ/cm² to about 10 mJ/cm². In one particular embodiment, the fracturing fluid containing D. desulfuricans is exposed to a dose of polychromatic UV radiation of about 7.5 mJ/cm².

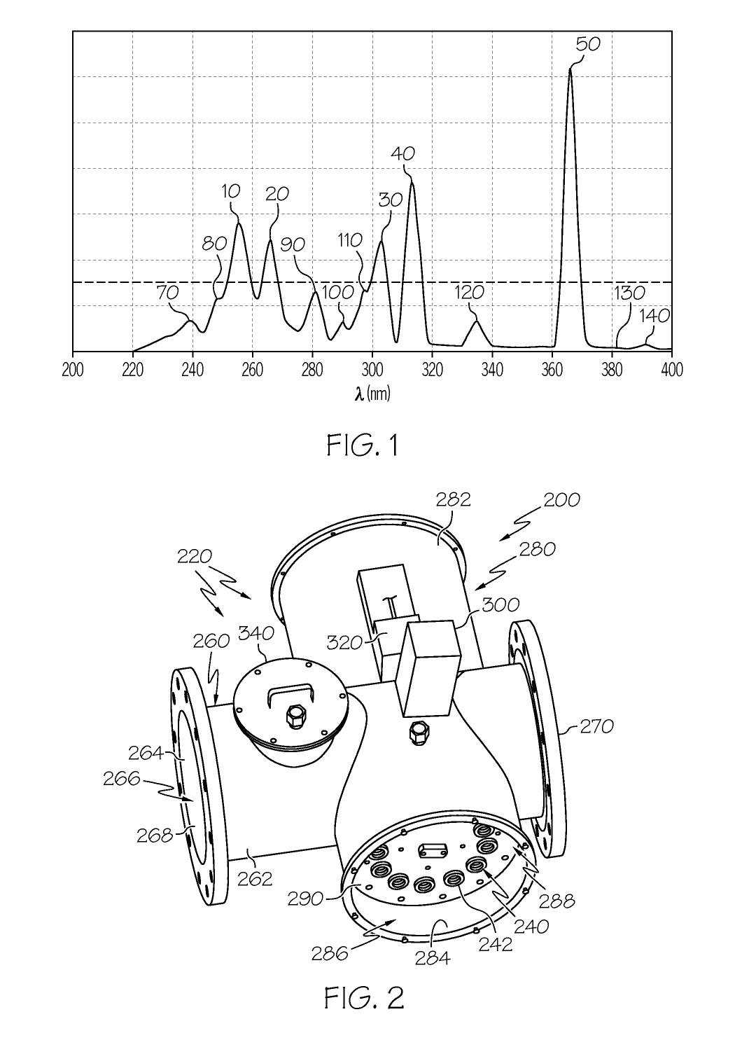

Referring to FIG. 1, the polychromatic UV radiation includes a plurality of major inactivation wavelength peaks (10, 20, 30, 40, 50) in the wavelength range of from about 200 nm to about 400 nm. A major inactivation wavelength peak is characterized by at least the following features: 1) an intensity greater than about 25% of a maximum peak intensity of the plurality of major inactivation wavelength peaks (as represented by the dashed line in FIGS. 1); and 2) a full width half maximum value greater than about 2 nm. The maximum peak intensity is characterized by the peak of the polychromatic UV radiation spectrum which has a full width half maximum value greater than about 2 nm and has the greatest intensity. For example, in FIG. 1, the maximum peak intensity is characterized by the intensity of major inactivation wavelength peak 50.

The polychromatic UV radiation includes at least five major inactivation wavelength peaks (10, 20, 30, 40, 50) in the wavelength range of from about 200 nm to about 400 nm. More particularly, the polychromatic UV radiation includes a plurality of major inactivation wavelength peaks (10, 20) in the wavelength range of from about 200 nm to about 300 nm, and a plurality of major inactivation wavelength peaks (30, 40, 50) in the wavelength range of from about 300 nm to about 400 nm.

In one particular embodiment, the polychromatic UV radiation includes three major inactivation wavelength peaks in the wavelength range of from about 200 nm to about 400 nm, having at least one major inactivation wavelength peak in the wavelength range of from about 200 nm to about 300 nm and at least one major inactivation wavelength peak in the wavelength range of from about 300 nm to about 400 nm. In another embodiment, the polychromatic UV radiation includes at least two major inactivation wavelength peaks in the wavelength range of from about 200 nm to about 300 nm, and at least two major inactivation wavelength peaks in the wavelength range of from about 300 nm to about 400 nm. In still another embodiment, the polychromatic UV radiation includes at least two major inactivation wavelength peaks in the wavelength range of from about 200 nm to about 300 nm, and at least three major inactivation wavelength peaks in the wavelength range of from about 300 nm to about 400 nm.

Still referring to FIG. 1, the polychromatic UV radiation includes a plurality of minor inactivation wavelength peaks (70, 80, 90, 100, 110, 120, 130, 140) in the wavelength range of from about 200 nm to about 400 nm. In one particular embodiment, the polychromatic UV radiation includes at least two minor inactivation wavelength peaks. In another embodiment, the polychromatic UV radiation includes a plurality of minor inactivation wavelength peaks (70, 80, 90, 100, 110) in the wavelength range of from about 200 nm to about 300 nm, and a plurality of minor inactivation wavelength peaks (120, 130, 140) in the wavelength range of from about 300 nm to about 400 nm. A minor inactivation wavelength peak is characterized by at least one of the following features: 1) an intensity less than or equal to about 25% of the maximum peak intensity of the plurality of major inactivation wavelength peaks (as represented by the dashed line in FIGS. 1); and 2) a full width half maximum value less than or equal to about 2 nm. The maximum peak intensity is characterized as previously discussed, e.g. by the peak of the polychromatic UV radiation spectrum which has a full width half maximum value greater than about 2 nm and has the greatest intensity. With regard to FIG. 1, the maximum peak intensity is characterized by the intensity of major inactivation wavelength peak 50.

The fracturing fluid flows substantially unidirectionally from a fracturing fluid source to a wellbore. The fracturing fluid source may include, but should not be limited to, tanks, receptacles, cisterns, chambers, reservoirs, vats, tubs, and/or barrels. However, the fracturing fluid source may include any object and/or location wherein a fracturing fluid is stored. As previously discussed, the method includes exposing the fracturing fluid containing D. desulfuricans to a dose of from about 4 mJ/cm² to about 10 mJ/cm² of polychromatic UV radiation. Referring to FIG. 2, the fracturing fluid is exposed to the polychromatic UV radiation via at least one medium pressure UV lamp 240. The medium pressure UV lamp 240 is arranged substantially transverse to the flow of the fracturing fluid. In one particular embodiment, the medium pressure UV lamp 240 is arranged substantially transverse to the flow of the fracturing fluid and in between the fracturing fluid source and the wellbore. In another embodiment, the wellbore is provided in a reservoir for the production of at least one of petroleum and natural gas. The medium pressure UV lamp 240 is discussed in greater detail in a later section.

II. System for Inactivating D. desulfuricans

In another embodiment, a system for inactivating D. desulfuricans in a fracturing fluid containing the D. desulfuricans is disclosed. The system may be employed to perform the methods for inactivating D. desulfuricans in a fracturing fluid containing the D. desulfuricans previously discussed. More particularly, the system may be employed in an oilfield installation and/or the system may be installed on a mobile trailer for oilfield service use to perform the methods for inactivating D. desulfuricans as previously discussed.

Referring to FIG. 2, the system 200 includes an UV radiation chamber 220 and at least one medium pressure UV lamp 240, wherein the medium pressure UV lamp 240 is arranged substantially within the UV radiation chamber 220. The UV radiation chamber 220 is in fluid communication with a fracturing fluid source (not shown) and a wellbore (not shown). The fracturing fluid flows substantially unidirectionally from the fracturing fluid source through the UV radiation chamber 220 to the wellbore. The medium pressure UV lamp 240 exposes the fracturing fluid containing the D. desulfuricans to a dose of from about 4 mJ/cm² to about 10 mJ/cm² of polychromatic UV radiation. The polychromatic UV radiation includes a plurality of major inactivation wavelength peaks in the wavelength range of from about 200 nm to about 400 nm. The dose of polychromatic UV radiation inactivates the D. desulfuricans.

The UV radiation chamber 220 includes a fracturing fluid portion 260 integral with a medium pressure UV lamp portion 280. The fracturing fluid portion 260 has an exterior surface 262 and an interior surface 264 and defines a channel 266 through which the fracturing fluid may flow. The fracturing fluid portion 260 may have, but should not be limited to, a substantially elongate shape. In one particular embodiment, the fracturing fluid portion 260 has a substantially cylindrical shape. The shape of the fracturing fluid portion 260 should not be limited to those disclosed herein, however, but may include any shape wherein a fracturing fluid may flow therethrough.

The channel 266 provides an inlet 268 through which the fracturing fluid may enter the UV radiation chamber 220 and an outlet 270 through which the fracturing fluid may exit the UV radiation chamber 220. It is understood by one of ordinary skill in the art that the inlet 268 and the outlet 270 may be transposed (i.e. the inlet 270 and the outlet 268) such that the fracturing fluid flows through the UV radiation chamber 220 in substantially the opposite direction. The channel 266 may have a substantially circular, oblong, or elliptical cross-sectional shape. In one particular embodiment, the channel 266 has a substantially circular cross-sectional shape. However, the cross-sectional shape of the channel 266 should not be limited to those disclosed herein, but may include any cross-sectional shape wherein the fracturing fluid may flow therethrough.

The medium pressure UV lamp portion 280 has an exterior surface 282 and an interior surface 284 and defines an inner space 286. The inner space 286 may have a substantially circular, oblong, or elliptical cross-sectional shape. In one particular embodiment, the inner space has a substantially circular cross-sectional shape. However, the cross-sectional shape of the inner space 286 should not be limited to those disclosed herein, but may include any cross-sectional shape wherein at least a portion of the medium pressure UV lamp 240 may be arranged.

In one embodiment, the inner space 286 is provided with at least one endcap 288 sized to fit securely within the inner space 286. More particularly, the inner space 286 is provided with a pair of endcaps 288 (only one endcap shown) which are complementary in size and shape to the inner space 286. When the endcaps 288 are installed within the inner space, the endcaps 288 are in direct contact with the interior surface 284 of the medium pressure UV lamp portion 280 such that the fracturing fluid does not exit the UV radiation chamber 220 through the medium pressure UV lamp portion 280. In this way, the endcaps 288 function to seal the UV lamp portion 280. It is understood by one of ordinary skill in the art, however, that additional seals may be installed within the UV lamp portion 280 to prevent the fracturing fluid from exiting the UV radiation chamber 220 through the medium pressure UV lamp portion 280.

The medium pressure UV lamp portion 280 intersects with and is substantially normal to the fracturing fluid portion 260. In this way, the UV radiation chamber 220 is substantially T-shaped. Additionally, the inner space 286 defined by the medium pressure UV lamp portion 280 intersects with and is substantially normal to the channel 266 defined by the fracturing fluid portion 260, such that the inner space 286 and the channel 266 are in fluid communication. The inner space 286 defined by the medium pressure UV lamp portion 280 and the channel 266 collectively form a UV radiation space (not shown), wherein the fracturing fluid flows and is exposed to a dose of polychromatic UV radiation.

The UV radiation chamber 220 is made from any structurally suitable material, including plastics, polymers, elastomers, metals, composites, alloys, minerals, or the like. In one embodiment, the UV radiation chamber 220 is made from stainless steel; more particularly, the UV radiation chamber 220 is made from grade 316 stainless steel. The UV radiation chamber 220 is designed such that there is no exposure to UV radiation from the medium pressure UV lamps 240. Moreover, the UV radiation chamber 220 is designed to withstand about 2500 psi pressure washing. In one particular embodiment, the empty weight of the UV radiation chamber 220 is about 375 lb and the maximum pressure of the UV radiation chamber 220 is about 150 psi.

The medium pressure UV lamp 240 is arranged substantially within the UV radiation chamber 220. More particularly, the medium pressure UV lamp 240 is arranged substantially within the inner space 286 defined by the medium pressure UV lamp portion 280. The medium pressure UV lamp 240 is substantially elongate. In this embodiment, the medium pressure UV lamp 240 has a longitudinal axis and is arranged substantially within the inner space 286 such that the medium pressure UV lamp 240 extends along the length of the medium pressure UV lamp portion 280. In this way, the longitudinal axis of the medium pressure UV lamp 240 is substantially transverse to the flow of the fracturing fluid.

The medium pressure UV lamp 240 provides an operating power of from about 2650 W to about 3800 W and a maximum power consumption of about 4.5 kW. In one embodiment, the medium pressure UV lamp 240 is a B3535 type lamp which provides an operating power of about 2.7 kW, or alternatively of about 3.1 kW, or alternatively of about 3.8 kW. The medium pressure UV lamp 240 has a life of about 4000 H.

In one embodiment, the medium pressure UV lamp 240 is protected from contact with the fracturing fluid by enclosing and/or covering the medium pressure UV lamp 240 with a protective sleeve 242. The sleeve 242 defines an inner space for accommodating the medium pressure UV lamp 240. The sleeve 242 may have a shape which is substantially the same as the shape of the medium pressure UV lamp 240. In one embodiment, the sleeve 242 has a substantially elongate shape and defines an inner space for accommodating the medium pressure UV lamp 240 therein. In another particular embodiment, the sleeve 242 has a substantially cylindrical shape and defines an inner space in which a medium pressure UV lamp 240 is enclosed. The protective sleeve 242 is made of high purity quartz.

When the medium pressure UV lamp 240 is enclosed by a sleeve 242, the sleeve 242 is arranged substantially within the inner space defined by the medium pressure UV lamp portion 280 such that the sleeve 242 extends along the length of the medium pressure UV lamp portion 280. In this way, the longitudinal axis of the sleeve 242 is substantially transverse to the flow of the fracturing fluid. In one particular embodiment, the sleeve 242 extends from one of the pair of endcaps 288 through the inner space 286 to the remaining endcap 288. Alternatively, in another embodiment, the sleeve 242 extends from an outer surface 290 of one of the pair of endcaps 288, through the endcap 288 to the inner space 286, through the inner space 286, and through the remaining endcap 288 to the outer surface 290 of the remaining endcap 288. In this way, the medium pressure UV lamp 240 which is arranged within the inner space defined by the sleeve 242, is accessible from the exterior of the UV radiation chamber 220, and more particularly from the inner space 286 of the medium pressure UV lamp portion 280, such that the medium pressure UV lamp 240 may be removed from and/or inserted within the sleeve 242 (and also the inner space 286 defined by the medium pressure UV lamp portion 280) without requiring that the UV radiation space be drained of the fracturing fluid.

In one embodiment, the system 200 includes a plurality of medium pressure UV lamps 240. For example, the system 200 includes at least two, or alternatively at least four, or alternatively at least six medium pressure UV lamps 240. In one particular embodiment, the system 200 includes twelve medium pressure UV lamps 240. When the system 200 includes a plurality of medium pressure UV lamps 240, each of the medium pressure UV lamps 240 may be enclosed by a sleeve 242, as previously discussed.

The fracturing fluid flows substantially unidirectionally from the fracturing fluid source through the UV radiation chamber 220 to the wellbore. More specifically, the fracturing fluid flows from the fracturing fluid source, through the inlet 268 of the UV radiation chamber 220, through the UV radiation space, and through the outlet 270 of the UV radiation chamber 220, to the wellbore. The bulk fluid movement of the fracturing fluid through the UV radiation chamber 220 is up to about three million gallons per day (i.e. MGD), or up to about 2100 gallons per minute (i.e. GPM). With regard to the flow of the fracturing fluid through the UV radiation chamber 220, the maximum allowable head loss of the fracturing fluid through the UV radiation chamber 220 is less than about 14 in at maximum flow.

The dose of polychromatic UV radiation and plurality of major inactivation wavelength peaks are as previously discussed.

In addition to the UV radiation chamber 220 and the medium pressure UV lamp 240, the system 200 may also include an UV intensity monitor 300, a temperature sensor 320, and an access hatch 340. The system 200 may also include any additional components not specifically discussed herein which would be useful in performing methods for inactivating Desulfovibrio desulfuricans in a fracturing fluid.

The medium pressure UV lamp 240 may be equipped with an UV intensity monitor 300. The UV intensity monitor 300 is in communication with the medium pressure UV lamp 240, such that the UV intensity monitor 300 may measure the UV intensity of each medium pressure UV lamp 240. In this way, continuous performance of each of the medium pressure UV lamps 240 may be monitored and verified. The UV intensity monitor 300 may be fitted with a filter (not shown), wherein the filter only monitors UV energy in the wavelength range of from about 220 nm to about 290 nm. In one embodiment, the UV intensity monitor 300 is protected from contact with the fracturing fluid (and any other fluids) by enclosing and/or covering the UV intensity monitor 300 with a protective housing. The housing may be made from any structurally suitable material, including plastics, polymers, elastomers, metals, composites, alloys, minerals, or the like. In one particular embodiment, the housing is made of stainless steel.

The UV intensity monitor 300 is in communication with the medium pressure UV lamp 240 via an intensity monitor site (not shown) in the exterior surface 282 of the medium pressure UV lamp portion 280. The intensity monitor site is made from high purity quartz. The UV intensity monitor 300 may be unaffected by static, electromagnetic fields, and/or short wave radio emissions that comply with current FCC regulations. The UV intensity monitor 300 may be in signal communication with a control module (not shown) and may produce a signal of from about 4 mA to about 20 mA, which may be sent to the control module. Alternatively, in another embodiment, the UV intensity monitor 300 may be electrically connected to the control module with a cable.

The temperature sensor 320 may be fitted to the UV radiation chamber 220 to monitor the temperature of the system 200. More particularly, the temperature sensor 320 may be fitted to the UV radiation chamber 220 to monitor the temperature to protect against temperature variation and/or buildup in situations wherein the flow of the fracturing fluid is low and/or halted. In one particular embodiment, the temperature sensor 320 may be fitted to the exterior surface 282 of the medium pressure UV lamp portion 280. The temperature sensor 320 may be protected from contact with the fracturing fluid (and any other fluids) by enclosing and/or covering the temperature sensor 320 with a protective housing. The housing may be made from any structurally suitable material, including plastics, polymers, elastomers, metals, composites, alloys, minerals, or the like. In one particular embodiment, the housing is made of stainless steel.

The access hatch 340 may be provided on the exterior surface 262 of the fracturing fluid portion 260. The access hatch 340 allows easy, simple access for visual inspection of the medium pressure UV lamps 240 and/or the sleeves 242. Moreover, the access hatch 340 allows for removal of foreign debris from the UV radiation space without requiring the removal of the medium pressure UV lamps 240 and/or sleeves 242. The access hatch 340 may have a substantially circular shape. However, the shape of the access hatch 340 should not be limited to circular, but may be any shape which provides access for visual inspection and/or for the removal of foreign debris from the UV radiation space. The access hatch 340 may be made from any structurally suitable material, including plastics, polymers, elastomers, metals, composites, alloys, minerals, or the like. In one particular embodiment, the access hatch 340 is made of stainless steel.

One of ordinary skill in the art will recognize that the components that make up the system 200 may be made from any structurally suitable material, including plastics, polymers, elastomers, metals, composites, alloys, minerals, or the like. For example, the components that contact the fracturing fluid (i.e. the wetted parts of the system 200) may be made from polytetrafluoroethylene (hereinafter “PTFE” or “Teflon®”), ethylene propylene diene monomer (hereinafter “EPDM”), stainless steel, and/or high purity quartz, and combinations thereof.

Additionally, in order to control the flow of the fracturing fluid, the system 200 may also include isolation valves (not shown). More specifically, the system 200 may include isolation valves installed upstream and/or downstream of the system 200 and in fluid communication with the UV radiation chamber 220, the fracturing fluid source, and the wellbore.

### EXAMPLES

The following non-limiting example illustrates the methods of the present disclosure.

### Example 1

### Inactivation of Desulfovibrio desulfuricans by Medium Pressure UV Light

Bacteria and Media Preparation. All work was performed by Clancy Environmental Consultants Inc. (St. Albans, Vt.). Desulfovibrio desulfuricans subsp. desulfuricans 29577 was acquired from American Type Culture Collection (Manassas, Va.) and 13 ˜1 mL aliquots of D. desulfuricans were obtained from the University of Oklahoma (Norman, Okla.). The 13 ˜1 mL aliquots of D. desulfuricans were grown in a sulfate-reducing bacteria (SRB) medium which is a modification from an API-RST and API RP-38 medium. D. desulfuricans was propagated in an anaerobic environment (see below) using a modified Baar's medium for sulfate reducers. Anaerobic pre-reduced modified Baar's medium was acquired from Anaerobe Systems. D. desulfuricans was enumerated on modified iron sulphite agar (mISA) (Mara and Williams, 1970.) An anaerobic environment was created using BD GasPak™ EZ Anaerobe Container and Pouch Systems from BD (Franklin Lakes, N.J.). These systems created an anaerobic environment (≦1% oxygen) within 2.5 hours when incubated at 35° C. As a verification of an anaerobic environment, an anaerobic indicator strip was added to all containers.

Propagation of D. desulfuricans. Propagation of D. desulfuricans followed the protocol as described by the American Type Culture Collection with a few modifications. The rehydrated pellet was not held under a stream of oxygen-free sterile gas when aseptically transferred to 0.5 mL of Baar's medium. Pre-reduced Baar's medium was extracted from the hungate tube with a sterile 1 mL syringe and used to rehydrate the pellet. Once the pellet was rehydrated, a sterile 1 mL syringe was used to return the inoculum to the pre-reduced Baar's medium and it was incubated anaerobically at 30° C. until a black precipitate formed. Once propagated, 1 mL aliquots in cryogenic vials with 300 μl of glycerol were stored in −80° C. freezer for long term storage.

Seeded Suspension Preparation for Irradiations. Initial plans called for evaluating the UV dose response of D. desulfuricans across a range of water quality, or UV transmittance (UVT), from about 10 to about 90% through 1 cm. However, it was noted that the transmittance of UVT of suspensions seeded to a concentration of 1×10⁶/mL was only 25% through 1 cm, precluding testing at higher water qualities. For each exposure, a 6 mL suspension was decanted into a petri dish, which was immediately placed in an irradiation chamber (see collimated beam procedures, below). The petri dish was stirred during irradiation with 2.5×12 mm stir bars.

Medium Pressure UV Dose Response. The UV source for the medium pressure collimated beam process was a medium pressure lamp (Rayox® 1 kW). This lamp was housed above a shutter. When the shutter was opened, light from the lamp passed through a collimating tube (92 cm) to irradiate test organisms suspended in a petri dish (6 cm diameter). Due to the anaerobic nature of the Desulfovibrio desulfuricans, the petri dish was placed in an irradiation chamber (65 mm diameter) that was supplied through a side port with nitrogen gas at a rate of 4 standard cubic feet per hour (hereinafter “SCFH”), to purge the environment of oxygen. The irradiation chamber was fitted with a quartz disk cover (70 mm diameter) that was transparent to UV light. Prior to irradiations, the lamp was allowed to warm up for over 30 minute.

The UV incident to the surface of the Petri dish was then measured using a radiometer and detector (International Light 1400/SED240/T2ACT5) using the sensitivity factor of the sensor derived by a special calibration designed to allow measurement of the germicidal UV emitted from the polychromatic medium pressure lamp. The incident irradiation across the surface of the Petri dish was measured at 5 mm intervals along an X-Y grid originating at the center of the dish. Radiometer readings were taken with the detector placed within the irradiation chamber, to account for any loss of irradiance through the quartz cover. Overall irradiance distribution was then determined relative to the center reading. This value was used in the calculation of average irradiation incident to the water surface. Factors influencing average irradiation to the entire volume include reflection from the water surface, depth of the water, and UV absorption of the inoculated test water. The latter was measured at 254 nm by spectrophotometry (Spectronic Genesys 10uv™). UV dose was defined as the irradiation multiplied by the exposure time.

Irradiations of the seeded batches of the bacteria were made by placing the petri dish into the irradiation chamber, under the center of the collimating tube. The UV lamp shutter was then opened and the suspension irradiated for the pre-determined length of time to produce a range of exposure times to provide UV doses of 0, 1, 2, 4, 6, and 10 mJ/cm². A 0 mJ/cm² dose was run simultaneously with the irradiation test for the highest mJ/cm² dose, but in the absence of UV. This 0 dose sample provides the base count for determination of log₁₀ inactivation. Duplicate exposures were run.

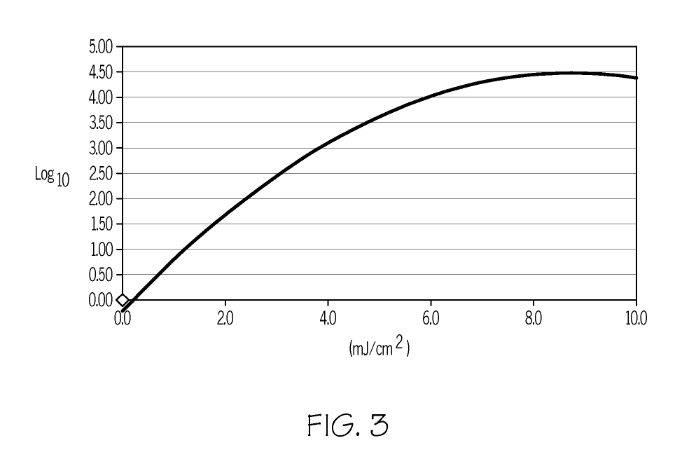

Sample Analysis. After exposure, samples were received in the lab in sterile 15 mL polypropylene centrifuge tubes labeled with their appropriate dose exposures. All samples were serially diluted by removing 1 mL of sample and injecting into pre-reduced 9 mL dilution blanks until the desired dilutions were achieved and then transferred into sterile 1.5 mL microcentrifuge tubes. All anaerobic pre-reduced 9 mL dilution blanks contain buffered mineral salts with sodium thioglycolate and L-cysteine added to provide a reduced environment. From each of these, 0.1 mL of sample was inoculated into an mISA tempered agar tube for enumeration by pour plate method. At least two and as many as three log dilutions of each sample were assayed. All dilutions were plated in triplicate and incubated at 30° C. in an anaerobic chamber for 5 days. Referring to FIG. 3, colony counts were then made, with each colony forming unit representing one surviving bacterium.

Results. As shown in FIG. 4 and Table 1 below, D. desulfuricans was found to be quite sensitive to UV disinfection. Additionally, as shown in Table 1, medium pressure UV was capable of inactivating D. desulfuricans by over 4 log₁₀.

TABLE 1
Inactivation of Desulfovibrio desulfuricans
by Medium Pressure UV Irradiation

| | Log₁₀ Inactivation | |
|---|---|---|
| UV Dose | Medium Pressure | |
| (mJ/cm²) | Replicate A | Replicate A |
| 0.0 | 0.0 | 0.0 |
| 0.5 | | |
| 1.0 | 0.6 | 0.5 |
| 2.0 | 1.6 | 1.4 |
| 3.0 | | |
| 4.0 | 3.6 | 3.4 |
| 5.0 | | |
| 6.0 | 4.1 | 4.0 |
| 10.0 | 4.4 | 4.3 |

The tailing of inactivation at or above 6 mJ/cm² is often seen in dose response studies, and without being bound by the theory, may have been exacerbated by the very low UV transmittance noted in the anaerobic bacterial suspensions. Ideal dose distributions generally approached by the use of stirred suspensions in collimated beam testing may be somewhat compromised.

Although some aspects of the present disclosure are identified herein as preferred or particularly advantageous, it is contemplated that the present disclosure is not necessarily limited to these preferred aspects of the disclosure.

For the purposes of describing and defining the present disclosure it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

It is noted that one or more of the following claims utilize the term “wherein” as a transitional phrase. It is noted that this term is introduced in the claims as an open-ended transitional phrase that is used to introduce a recitation of a series of characteristics of the structure and should be interpreted in like manner as the more commonly used open-ended preamble term “comprising.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are incorporated herein by reference; the citation of any document is not to be construed as an admission that it is prior art with respect to the present disclosure.

What is claimed is:

1. A method for inactivating Desulfovibrio desulfuricans in a fracturing fluid containing the Desulfovibrio desulfuricans, the method comprising:
exposing the fracturing fluid containing the Desulfovibrio desulfuricans to a dose of from about 4 mJ/cm² to about 10 mJ/cm² of polychromatic ultraviolet radiation, wherein the polychromatic ultraviolet radiation comprises a plurality of major inactivation wavelength peaks in a range of from about 200 nm to about 400 nm, wherein each of the major inactivation wavelength peaks is characterized by an intensity greater than about 25% of a maximum peak intensity of the plurality of major inactivation wavelength peaks and by a full width half maximum value greater than about 2 nanometers, and wherein the dose of polychromatic ultraviolet radiation inactivates the Desulfovibrio desulfuricans.

2. The method of claim 1, wherein the dose of polychromatic ultraviolet radiation is from about 6 mJ/cm² to about 10 mJ/cm².

3. The method of claim 1, wherein the dose of polychromatic ultraviolet radiation is from about 7.5 mJ/cm² to about 10 mJ/cm².

4. The method of claim 1, wherein the polychromatic ultraviolet radiation comprises a plurality of major inactivation wavelength peaks in the range of from about 200 nm to about 300 nm, and a plurality of major inactivation wavelength peaks in the range of from about 300 nm to about 400 nm.

5. The method of claim 1, wherein the polychromatic ultraviolet radiation comprises at least three major inactivation wavelength peaks in the range of from about 200 nm to about 400 nm, having at least one major inactivation wavelength peak in the range of from about 200 nm to about 300 nm, and at least one major inactivation wavelength peak in the range of from about 300 nm to about 400 nm.

6. The method of claim 1, wherein the polychromatic ultraviolet radiation comprises at least two major inactivation wavelength peaks in the range of from about 200 nm to about 300 nm and at least two major inactivation wavelength peaks in the range of from about 300 nm to about 400 nm.

7. The method of claim 1, wherein the polychromatic ultraviolet radiation comprises at least two major inactivation wavelength peaks in the range of from about 200 nm to about 300 nm and at least three major inactivation wavelength peaks in the range of from about 300 nm to about 400 nm.

8. The method of claim 1, wherein the polychromatic ultraviolet radiation comprises at least five major inactivation wavelength peaks.

9. The method of claim 1, wherein the polychromatic ultraviolet radiation comprises a plurality of minor inactivation wavelength peaks in a range of from about 200 nm to about 400 nm, wherein each of the minor inactivation wavelength peak is characterized by an intensity of less than or equal to about 25% a maximum peak intensity of the plurality of major inactivation wavelength peaks and by a full width half maximum value less than or equal to about 2 nanometers.

10. The method of claim 9, wherein the polychromatic ultraviolet radiation comprises at least two minor inactivation wavelength peaks.

11. The method of claim 1, wherein the fracturing fluid flows substantially unidirectionally from a fracturing fluid source to a wellbore, and wherein at least one medium pressure ultraviolet lamp is arranged substantially transverse to the flow of the fracturing fluid and exposes the fracturing fluid to the dose of polychromatic ultraviolet radiation.

12. The method of claim 1, wherein the fracturing fluid flows substantially unidirectionally from a fracturing fluid source to a wellbore, wherein at least one medium pressure ultraviolet lamp is arranged substantially transverse to the flow of the fracturing fluid and in between the fracturing fluid source and the wellbore, and wherein the at least one medium pressure ultraviolet lamp exposes the fracturing fluid to the dose of polychromatic ultraviolet radiation.

13. The method of claim 11, wherein the wellbore is provided in a reservoir for the production of at least one of oil and natural gas.

14. The method of claim 1, wherein the fracturing fluid comprises water.

15. A method for inactivating Desulfovibrio desulfuricans in a fracturing fluid containing the Desulfovibrio desulfuricans, the method comprising:
exposing the fracturing fluid containing the Desulfovibrio desulfuricans to a dose of from about 4 mJ/cm² to about 10 mJ/cm² of polychromatic ultraviolet radiation, wherein the fracturing fluid comprises water, wherein the polychromatic ultraviolet radiation comprises at least five inactivation wavelength peaks in a range of from about 200 nm to about 400 nm, wherein each of the major inactivation wavelength peaks is characterized by an intensity greater than about 25% of a maximum peak intensity of the plurality of major inactivation wavelength peaks and by a full width half maximum value greater than about 2 nanometers, wherein the dose of polychromatic ultraviolet radiation inactivates the Desulfovibrio desulfuricans, and wherein the fracturing fluid flows substantially unidirectionally from a fracturing fluid source to a wellbore.

16. A system for inactivating Desulfovibrio desulfuricans in a fracturing fluid containing the Desulfovibrio desulfuricans, the system comprising:
an ultraviolet radiation chamber in fluid communication with a fracturing fluid source and a wellbore, wherein the fracturing fluid flows substantially unidirectionally from the fracturing fluid source through the ultraviolet radiation chamber to the wellbore; and
at least one medium pressure ultraviolet lamp arranged substantially within the ultraviolet radiation chamber, wherein the medium pressure ultraviolet lamp exposes the fracturing fluid containing the Desulfovibrio desulfuricans to a dose of from about 4 mJ/cm² to about 10 mJ/cm² of polychromatic ultraviolet radiation, wherein the polychromatic ultraviolet radiation comprises a plurality of major inactivation wavelength peaks in a range of from about 200 nm to about 400 nm, wherein each of the major inactivation wavelength peaks is characterized by an intensity greater than about 25% a maximum peak intensity of the plurality of major inactivation wavelength peaks and by a full width half maximum value greater than about 2 nanometers, and wherein the dose of polychromatic ultraviolet radiation inactivates the Desulfovibrio desulfuricans.

17. The system of claim 16, wherein a plurality of medium pressure ultraviolet lamps are arranged substantially within the ultraviolet radiation chamber.

18. The system of claim 16, wherein a plurality of medium pressure ultraviolet lamps having longitudinal axes are arranged substantially within the ultraviolet radiation chamber such that the longitudinal axes are substantially transverse to the flow of the fracturing fluid.
